(19) United States
(12) Patent Application Publication
(10) Pub. No.: US 2013/0164833 A1
(43) Pub. Date: Jun. 27, 2013

(54) BIO-CHIP AND METHOD OF REPLACING CULTURE MEDIUM THEREOF

(71) Applicants: Dong Woo LEE, Suwon (KR); Sang Jin Kim, Hwaseong (KR); Bo Sung Ku, Suwon (KR)

(72) Inventors: Dong Woo LEE, Suwon (KR); Sang Jin Kim, Hwaseong (KR); Bo Sung Ku, Suwon (KR)

(73) Assignee: SAMSUNG ELECTRO-MECHANICS CO., LTD.

(21) Appl. No.: 13/409,990

(22) Filed: Mar. 1, 2012

(30) Foreign Application Priority Data
Dec. 21, 2011 (KR) 10-2011-0138827

(51) Int. Cl. C12M 1/00

(52) U.S. Cl. 435/305.2; 137/1

(57) ABSTRACT

There is provided a bio-chip including a substrate member including a plurality of recesses formed therein to accommodate a culture medium; and space maintaining members formed on the substrate member and maintaining a space between the substrate member and another substrate member to allow a bio-material in the culture medium to be transferred to a culture medium of another substrate member.

### CROSS-REFERENCE TO RELATED APPLICATIONS

This application claims the priority of Korean Patent Application No. 10-2011-0138827 filed on Dec. 21, 2011, in the Korean Intellectual Property Office, the disclosure of which is incorporated herein by reference.

### BACKGROUND OF THE INVENTION

1. Field of the Invention

The present invention relates to a bio-chip and a method of replacing a culture medium thereof, and more particularly, to a bio-chip allowing a culture medium to be easily replaced and a method of replacing a culture medium thereof.

2. Description of the Related Art

Recently, demand for bio-medical devices and bio-technology for quickly diagnosing various human diseases has increased. In line with this, the development of experimental devices and instruments that can show test results within a short time, instead of existing hospital or laboratory tests for a particular disease, requiring a long period of time, has been actively ongoing.

Meanwhile, the cultivation of an experimental target (e.g., a bio-material including cells) is essential for developing new medicines and experimenting with regard to the stability thereof. In general, the cultivation of a bio-material is undertaken in a culture container or a culture plate storing a culture medium.

Here, the culture medium of the culture plate is changed after the lapse of a certain amount of time or no longer reacts to a bio-material. Thus, in order to smoothly cultivate a bio-material, the culture medium should be replaced at an interval of a certain period or the bio-material should be moved to a new culture plate.

In the culture medium replacement methods, the latter is a method of only collecting a bio-material and moving it, so its operation is relatively easy in comparison with the former method of removing the culture medium and injecting a new culture medium.

However, the latter has a problem in which the bio-material is spoiled while being moved by using a tool such as a pincette, or the like.

### SUMMARY OF THE INVENTION

An aspect of the present invention provides a bio-chip allowing a culture medium of a bio-material to be easily replaced, and a method of replacing a culture medium thereof.

According to an aspect of the present invention, there is provided a bio-chip including: a substrate member including a plurality of recesses formed therein to accommodate a culture medium; and space maintaining members formed on the substrate member and maintaining a space between the substrate member and another substrate member to allow a bio-material in the culture medium to be transferred to a culture medium of another substrate member.

The space maintaining members may be asymmetrical with respect to a bisecting line of the substrate member in a first length direction or a second length direction thereof.

Each space maintaining member may include: a first portion having a convex shape; and a second portion having a concave shape to be engaged with the first portion having the convex shape.

Each recess may include: a bottom face coated with a hydrophilic material; and a side face coated with a hydrophobic material.

The space maintaining members may be disposed in a circular manner, based on the recesses.

According to another aspect of the present invention, there is provided a bio-chip including: a substrate member including a plurality of recesses formed therein to accommodate a culture medium; and an auxiliary member including a plurality of insertion pillars inserted into the recesses to allow the culture medium to be raised upwardly from the recesses, and through holes connected to the recesses.

Each recess may have a bottom face coated with a hydrophilic material.

The auxiliary member may be coated with a hydrophobic material.

Each insertion pillar may have a cylindrical shape having a hole connected to each recess.

Each insertion pillar may have a cross-sectional shape the same as that of each recess.

Each insertion pillar may be coated with a hydrophobic material.

Each insertion pillar may have a sloped face formed at an end thereof.

According to another aspect of the present invention, there is provided a method of replacing a culture medium of a bio-chip, the method including: preparing a first bio-chip accommodating a first culture medium, an old culture medium, therein and a second bio-chip accommodating a second culture medium, a new culture medium, therein; vertically inverting the first bio-chip to allow a bio-material included in the first culture medium to move out of a surface of the first culture medium; allowing the first and second bio-chips to approach nearer to each other such that the first and second culture mediums are brought into contact with each other; and separating the first and second bio-chips when the bio-material moves to the second culture medium through contact between the culture mediums.

The method may further include: vibrating the first bio-chip such that the bio-material in the first culture medium easily moves into the second culture medium.

The method may further include: disposing a space maintaining unit between the first and second bio-chips such that a predetermined distance is maintained between the first and second bio-chips.

According to another aspect of the present invention, there is provided a method of replacing a culture medium of a bio-chip, the method including: preparing a first bio-chip including a first recess formed therein and accommodating a first culture medium, an old culture medium, and a second bio-chip including a second recess formed therein and accommodating a second culture medium, a new culture medium; reducing an effective volume of the first recess such that the first culture medium is raised upwardly from a surface of the first bio-chip; vertically inverting the first bio-chip such that the bio-material included in the first culture medium moves out of a surface of the first culture medium; allowing the first and second bio-chips to approach nearer to each other such that the first and second culture mediums are brought into contact with each other; and separating the first and second bio-chips when the bio-material moves to the second culture medium through contact between the culture mediums.

In the reducing of the effective volume of the first recess, an auxiliary member having an insertion member inserted into the first recess and a through hole connected to the first recess may be stacked on the first bio-chip.

The method may further include: reducing an effective volume of the second recess such that the second culture medium of the second bio-chip is raised upwardly from a surface of the second bio-chip.

In the reducing of the effective volume of the second recess, an auxiliary member having an insertion member inserted into the second recess and a through hole connected to the second recess may be stacked on the second bio-chip.

According to another aspect of the present invention, there is provided a method of replacing a culture medium of a bio-chip, the method including: preparing a first bio-chip including a first recess formed therein and accommodating a first culture medium, an old culture medium, and a second bio-chip including a second recess formed therein and accommodating a second culture medium, a new culture medium; coupling an auxiliary member having an insertion pillar to be inserted into the second recess to the second bio-chip such that the second culture medium is raised upwardly from a surface of the second bio-chip; vertically inverting the first bio-chip such that the bio-material included in the first culture medium moves out of a surface of the first culture medium; allowing the first and second bio-chips to approach nearer to each other such that the first and second culture mediums are brought into contact with each other; and separating the first and second bio-chips when the bio-material moves to the second culture medium through contact between the culture mediums.

### BRIEF DESCRIPTION OF THE DRAWINGS

The above and other aspects, features and other advantages of the present invention will be more clearly understood from the following detailed description taken in conjunction with the accompanying drawings, in which:

FIGS. 1 and 2 are perspective views of a bio-chip according to a first embodiment of the present invention;

FIG. 3 is a perspective view of a bio-chip according to a second embodiment of the present invention;

FIG. 4 is a coupled cross-sectional view of the bio-chip illustrated in FIG. 3;

FIG. 5 is a perspective view of a bio-chip according to a third embodiment of the present invention;

FIG. 6 is a coupled cross-sectional view of the bio-chip illustrated in FIG. 5;

FIG. 7 is a view showing a method of replacing a culture medium of the bio-chip according to the first embodiment of the present invention; and

FIGS. 8 through 10 are views, each showing a method of replacing a culture medium of the bio-chip according to the second embodiment of the present invention.

### DETAILED DESCRIPTION OF THE EMBODIMENT

In describing the present invention below, terms indicating components of the present invention are named in consideration of functions of respective components. Therefore, the terms should not be understood as being limited technical components of the present invention.

For reference, a substrate used in the embodiment is not particularly limited and may be made of, for example, silicon, glass, metal, or polymer.

Types of polymer may include, for example, polymethylmethacrylate (PMMA), polycarbonate (PC), polystyrene (PS), polypropylene, cyclic olefin copolymer, polynorbonene, styrene-butadiene-copolymer (SBC), or acrylonitrile butadiene styrene, but the present invention is not limited thereto.

Also, a method of fabricating the substrate is not particularly limited. For example, the substrate may be fabricated through a photoresist process, an etching process, an injection-molding process, or the like.

In addition, a bio-material accommodated in the substrate mentioned in the specification may be used to include various materials including cells. For example, the bio-material may be a nucleic acid array such as RNA, DNA, or the like, peptide, protein, fat, an organic or inorganic chemical molecule, a virus particle, a prokaryotic cell, an organelle, or the like. Also, the bio-material may be used to include various animal cells or plant cells, rather than being limited to human cells.

The present invention may provide a bio-chip allowing a culture medium to be easily replaced in order to allow a bio-material to be smoothly cultivated, and a method of replacing a culture medium thereof.

In general, replacing a culture medium of a bio-material is performed by individually collecting bio-materials included in an old culture medium and moving the collected bio-materials to a new culture medium.

However, this method requires a great amount of time to collect the bio-materials, and the bio-materials may be damaged in the collection process thereof.

Also, since a time required for replacing a culture medium of a bio-material may be greatly varied in accordance with the order of collecting bio-materials, although the bio-materials are cultivated in the same bio-chip, different experiment results may be obtained.

Therefore, the present invention may provide a bio-chip in which a bio-material is transferred as a culture medium is brought into contact therewith to thereby allow the culture medium of the bio-material accommodated in the bio-chip to be simultaneously replaced, and a method of replacing a culture medium.

Embodiments of the present invention will be described in detail with reference to the accompanying drawings.

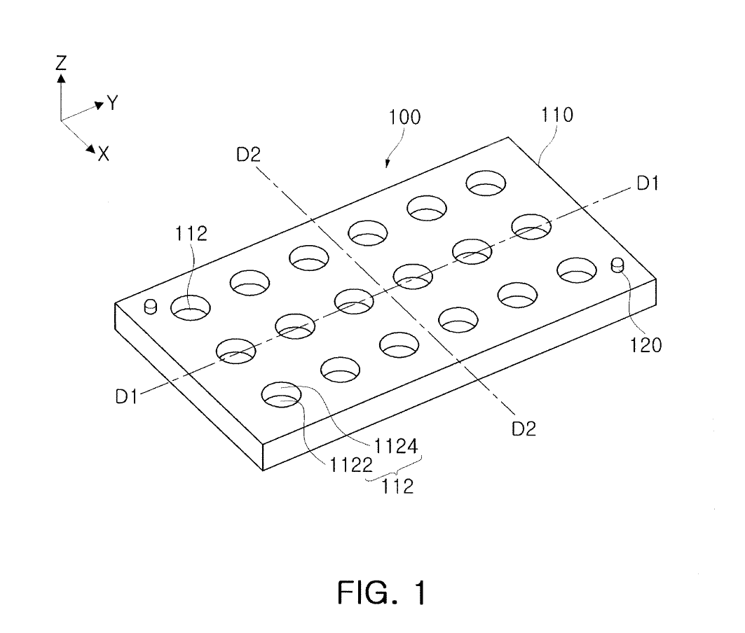

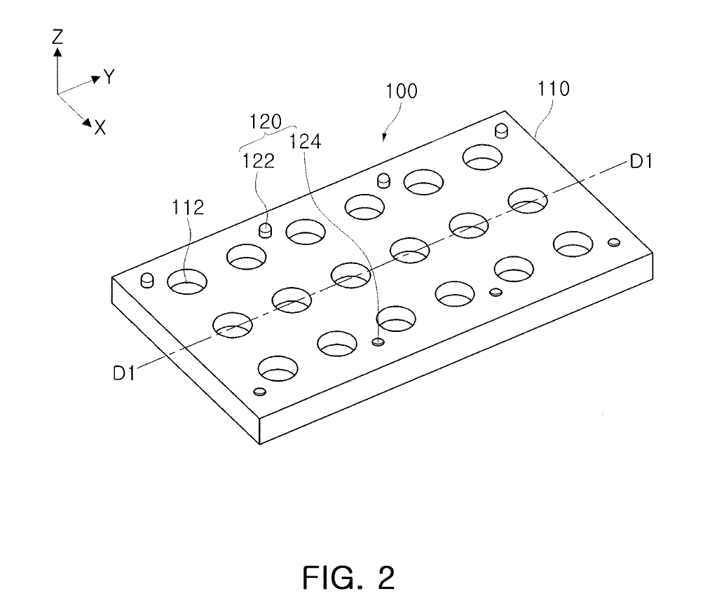

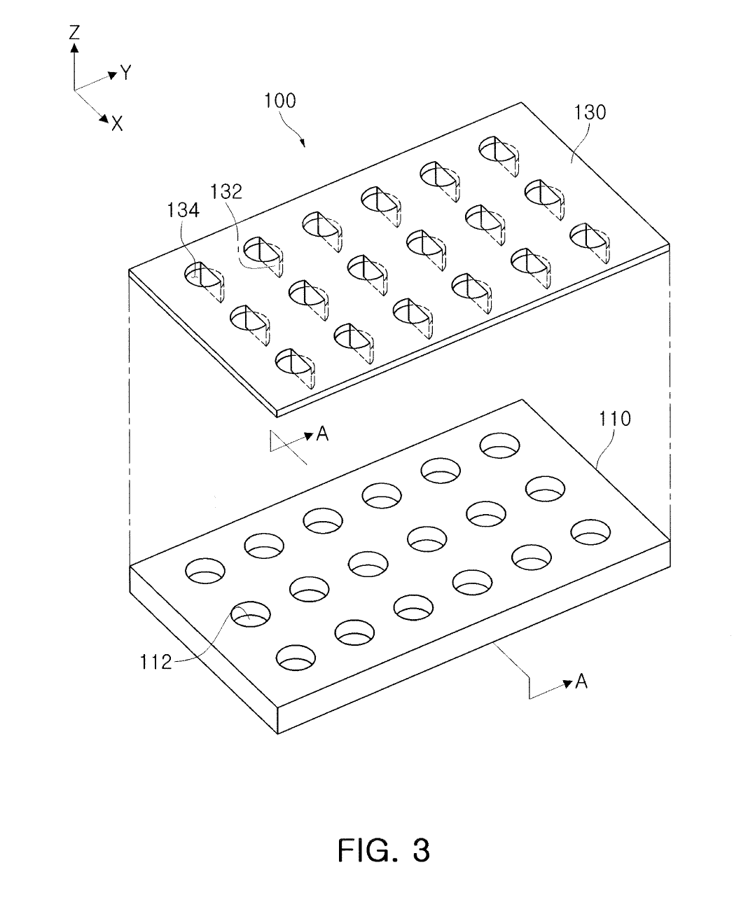

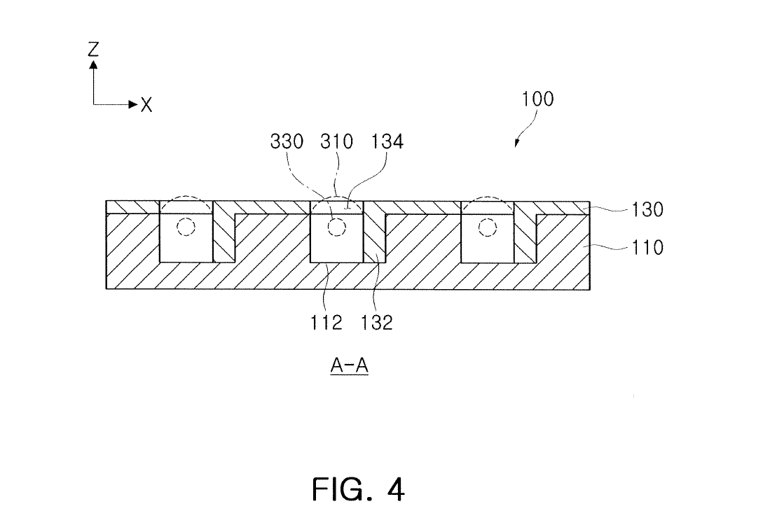

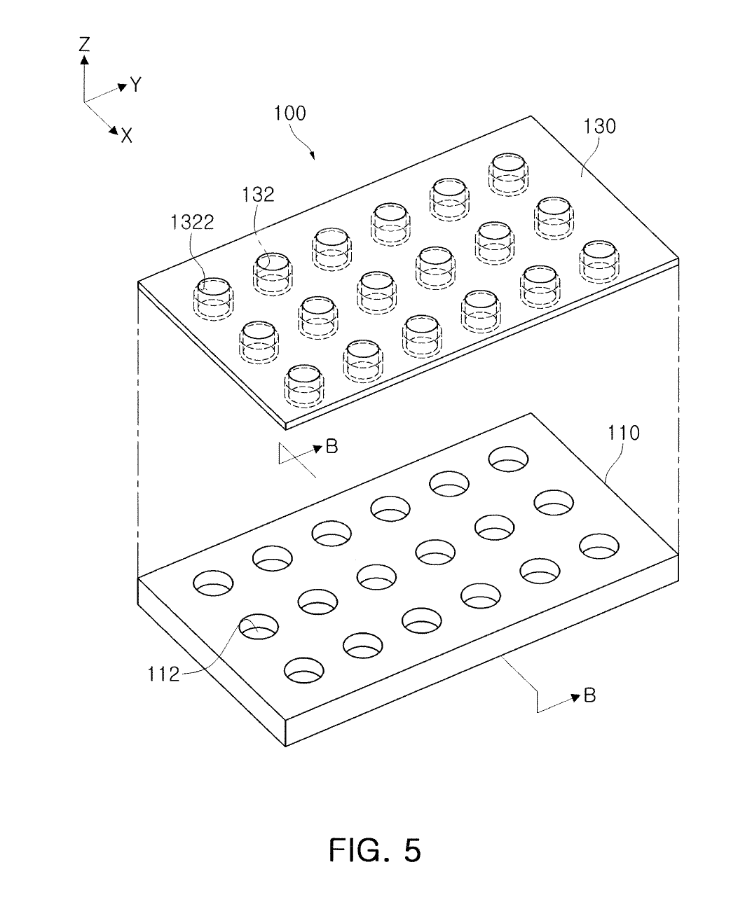

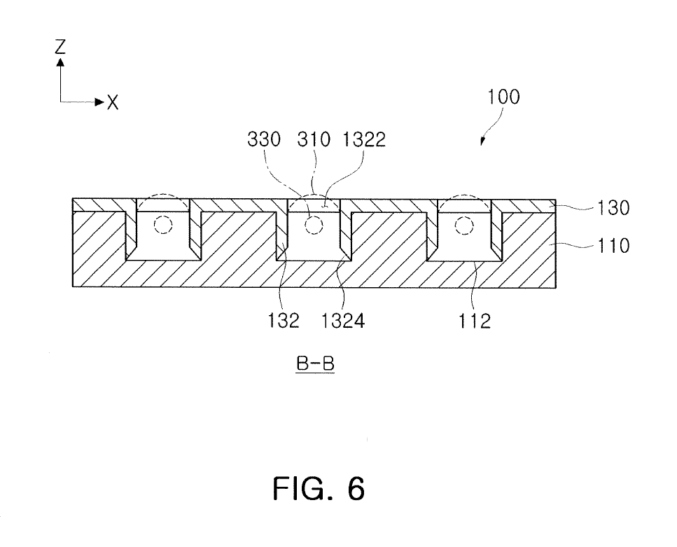

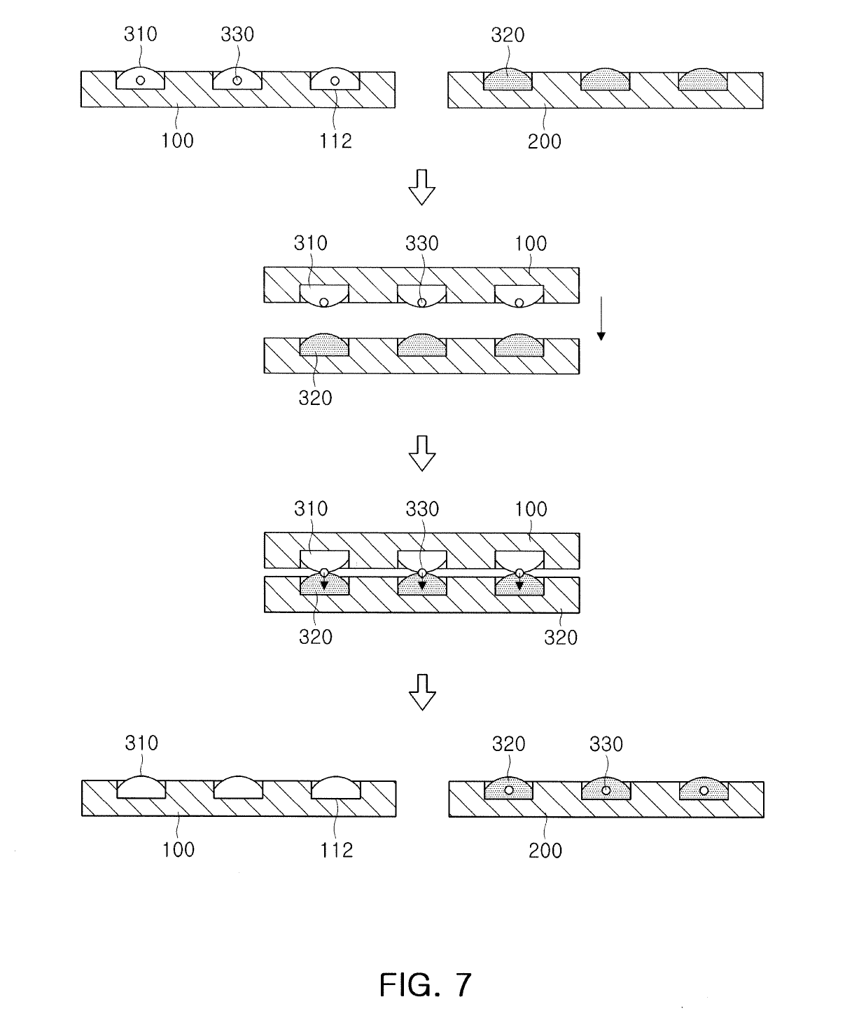

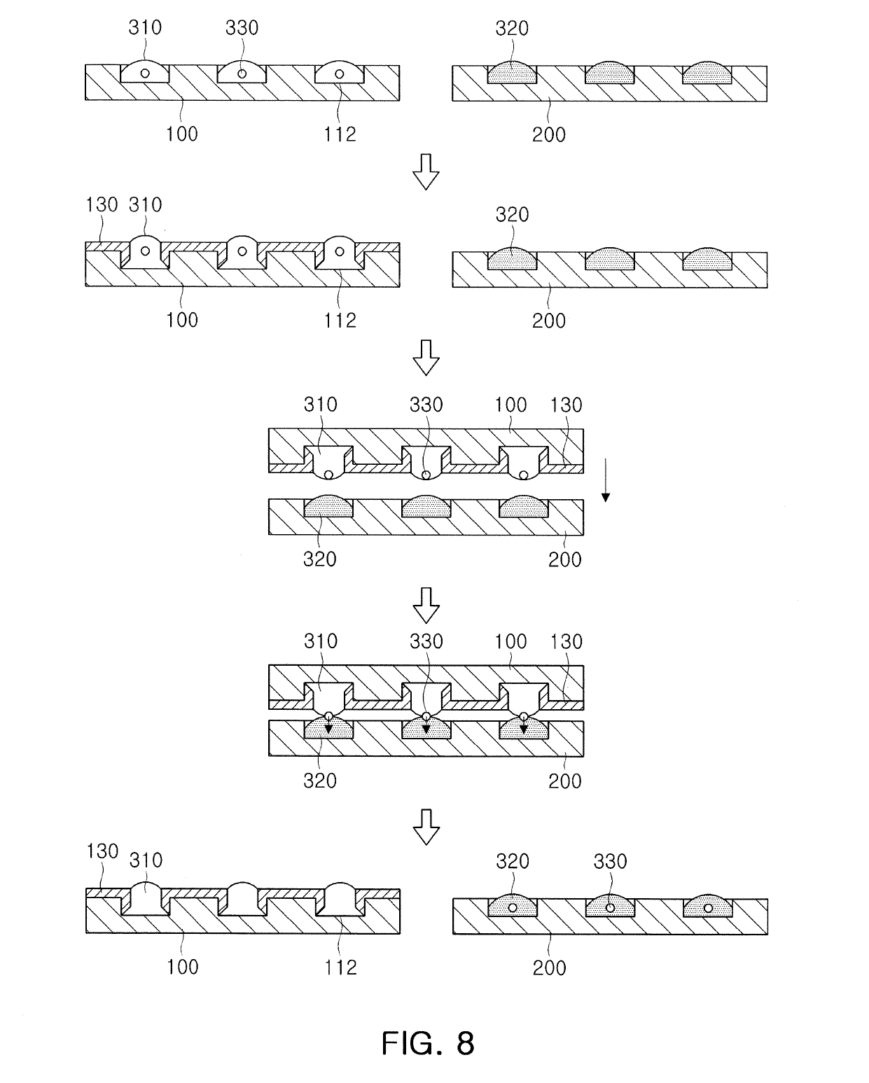

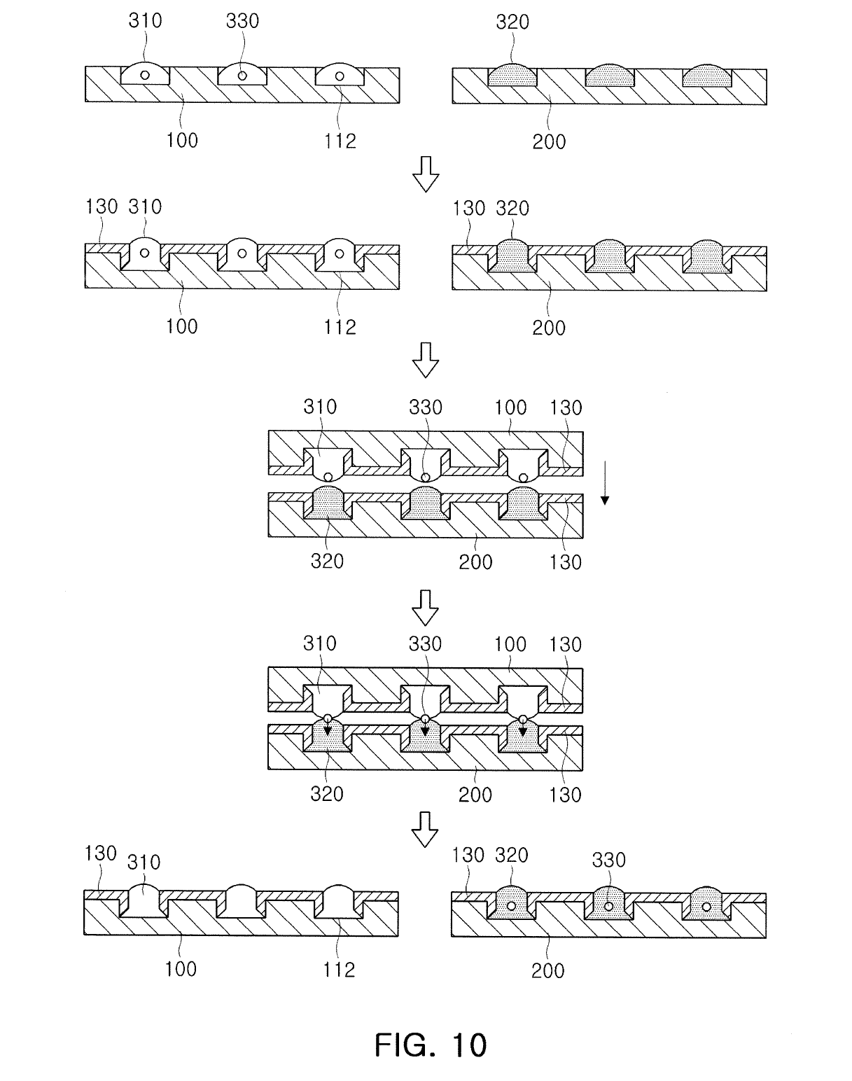

FIGS. 1 and 2 are perspective views of a bio-chip according to a first embodiment of the present invention. FIG. 3 is a perspective view of a bio-chip according to a second embodiment of the present invention. FIG. 4 is a coupled cross-sectional view of the bio-chip illustrated in FIG. 3. FIG. 5 is a perspective view of a bio-chip according to a third embodiment of the present invention. FIG. 6 is a coupled cross-sectional view of the bio-chip illustrated in FIG. 5. FIG. 7 is a view showing a method of replacing a culture medium of the bio-chip according to the first embodiment of the present invention. FIGS. 8 through 10 are views, each showing a method of replacing a culture medium of the bio-chip according to the second embodiment of the present invention.

A bio-chip 100 according to a first embodiment of the present invention may include a substrate member 110 and space maintaining members 120.

The substrate member 110 may be substantially a thin plate member having a rectangular shape. However, a cross-section of the substrate member 110 may have a square shape or a circular shape, rather than being limited to the rectangular shape.

The substrate member 110 may be made of a plastic material. The substrate member 110 made of a plastic material may be mass-produced through injection-molding, so manufacturing costs thereof may be reduced in comparison to the case of a bio-chip made of a glass material. In addition, the substrate member 110 made of a plastic material is relatively lightweight and has relatively low brittleness in comparison to the bio-chip made of glass material, and accordingly, it may be easily handled and has a low damage generation rate due to careless handling.

However, the substrate member 110 is not limited thereto. That is, for example, the substrate member 110 may be made of polymethylmethacrylate (PMMA), polycarbonate (PC), polystyrene (PS), cyclic olefin copolymer, polynorbonene, styrene-butadiene-copolymer (SBC), or acrylonitrile butadiene styrene.

A plurality of recesses 112 may be formed in a surface of the substrate member 110.

The recesses 112 may be formed in a plurality of rows in a first length direction (an X-axis direction based on FIG. 1) and a second length direction (Y-axis direction based on FIG. 1) of the substrate member 110. Also, the recesses 112 may have a predetermined depth in a thickness direction (a Z-axis direction based on FIG. 1) of the substrate member 110. The recesses 112 formed as above may accommodate a liquid culture medium and a bio-material therein.

Meanwhile, in FIG. 1, each of the recesses 112 has a circular cross-sectional shape, but the cross-sectional shape of the recesses 112 may be varied according to types of experiment targets and types of experiments. For example, each of the recesses 112 may have a square or polygonal cross-sectional shape.

Each recess 112 may have a bottom face 1122 and a side face 1124.

The bottom face 1122 may be coated with a hydrophilic material. The bottom face 1122 coated with a hydrophilic material may have excellent affinity with a bio-material or a culture medium, thereby enhancing stability of the bio-material or the culture medium against external impacts.

On the contrary, the side face 1124 may be coated with a hydrophobic material allowing the culture medium or the bio-material accommodated in the recess 112 to agglomerate into a water droplet. The side face 1124 coated with a hydrophobic material has low affinity with the bio-material or the culture medium, thereby preventing the bio-material or the culture medium from spreading widely.

The structure of the recess 112 may allow the bio-material or the culture medium to be maintained in a hemispherical shape, thus reducing an evaporation phenomenon of the bio-material or the culture medium and enhancing test reliability in scan test.

Each recess 112 may accommodate a bio-material and a culture medium therein. Here, the culture medium may be formed to have an upwardly-convex hemispherical shape, and may protrude upwardly from the surface of the substrate member 110.

The space maintaining members 120 may be formed on one surface of the substrate member 110. Each space maintaining member 120 may have a protrusion shape protruding from the surface of the substrate member 110.

The space maintaining members 120 may be formed to be asymmetrical to each other on the substrate member 110. For example, the space maintaining members 120 maybe formed to be asymmetrical with respect to a bisecting line D1-D1 of the substrate member 110 in the first length direction, or may be formed to be asymmetrical with respect to a bisecting line D2-D2 of the substrate member 110 in the second length direction.

As shown in FIG. 2, the space maintaining members 120 may include two portions 122 and 124 having different shapes. The first portions 122 each may have a cylindrical shape with a convex end, and the second portions 124 each may have a shape with a concave end. The first and second portions 122 and 124 maybe disposed to be symmetrical on the substrate member 110. For example, in FIG. 2, the first portions 122 and the second portions 124 may be symmetrical with respect to the bisecting line D1-D1.

The space maintaining members 120 formed above may uniformly maintain a distance between the recesses 112 when two bio-chips are stacked such that they face each other.

In detail, the space maintaining members 120 may allow a first culture medium of a first bio-chip and a second culture medium of a second bio-chip to be in contact within a limited range, and allow only bio-materials to be transferred through the contact between the culture mediums.

Bio-chips according to second and third embodiments of the present invention will be described with reference to FIGS. 3 through 6.

The bio-chip 100 according to a second embodiment of the present invention is different from that of the first embodiment, in that it further includes an auxiliary member 130.

A fixed amount of culture medium may be accommodated in the recesses 112 of the bio-chip 100 through an experimental instrument. Here, the fixed amount of culture medium may be sufficient to protrude upwardly from the surface of the substrate member 110, or may be insufficient to protrude upwardly from the surface of the substrate member 110 according to experiment types.

In consideration of this, the embodiment may further include the auxiliary member 130 which is able to reduce the volume of the recesses 112.

The auxiliary member 130 may have the same cross-sectional shape as that of the substrate member 110. However, the auxiliary member 130 may have a cross-sectional shape larger or smaller than that of the substrate member 110 as necessary.

The auxiliary member 130 may be coated with a hydrophobic material, which may minimize a phenomenon in which a culture medium or a bio-material is stuck to the auxiliary member 130.

The auxiliary member 130 may include insertion pillars 132 which may be inserted into the recesses 112 of the substrate member 110 and through holes 134 connected to the recesses 112.

The insertion pillars 132 may have a shape in which insertion pillars 132 be inserted into the recesses 112 in a state in which the substrate member 110 and the auxiliary member 130 are coupled. For example, each insertion pillar 132 may have a columnar shape with a semicircular cross-section. However, the shape of the insertion pillar 132 is not limited thereto and the insertion pillar 132 may have any shape so long as it can reduce the volume of each recess 112.

The through holes 134 may be connected to the recesses 112 in a state in which the substrate member 110 and the auxiliary member 130 are coupled. Namely, as shown in FIG. 4, the through holes 134 each may have an open shape such that a culture medium 310 of the recesses 112 is raised upwardly from the surface of the auxiliary member 130.

In the bio-chip 100 configured as described above, the volume of the recesses 112 may be reduced by the auxiliary member 130, so the culture medium 310 may easily protrude upwardly from the surface of the substrate member 110 or the auxiliary member 130.

Accordingly, the culture medium of the bio-material may be easily replaced through a contact of the culture medium (For reference, a method of replacing a culture medium using the same will be described later with reference to FIGS. 8 through 10).

Here, reference numeral 330 denotes a bio-material.

The bio-chip 100 according to the third embodiment of the present invention may be different from that of the second embodiment, in terms of the shape of the auxiliary member 130.

As shown in FIGS. 5 and 6, the auxiliary member 130 may include the insertion pillars 132 each having a substantially cylindrical shape.

Each insertion pillar 132 may have a cross-sectional shape which is substantially equal or similar to that of each recess 112. For example, the cross-sectional shape of each insertion pillar 132 may be a circle having the same diameter as that of the recess 112.

Each insertion pillar 132 may have a hole 1322 connected in the thickness direction (Z-axis direction based on FIG. 1 of the auxiliary member 130. Also, a sloped face 1324 may be formed at the end of the insertion pillar 132 as shown in FIG. 6.

The hole 1322 may be connected to each recess 112 in a state in which the substrate member 110 and the auxiliary member 130 are coupled. The hole 1322 may have a cross-sectional shape similar to that of the recess 112, and may have a cross-sectional area smaller than that of the recess 112. Thus, when each insertion pillar 132 is inserted into each recess 112, an effective volume of the recess 112 maybe reduced to a degree equal to a difference in the cross-sectional area between the recess 112 and the hole 1322.

In the bio-chip 100 configured as described above, since each insertion pillar 132 may allow the culture medium 310 to be collected toward the center of each recess 112, the culture medium 310 may be easily formed to have a hemispherical shape and the bio-material 330 maybe easily collected in the center of the recess 112.

A method of replacing a culture medium of the bio-chip according to the first embodiment of the present invention will be described with reference to FIG. 7.

The method of replacing a culture medium of the bio-chip according to the first embodiment of the present invention may include preparing first and second bio-chips, inverting the first bio chip, and bonding and separating the first and second bio-chips.

(Preparing First and Second Bio-Chips)

In this operation, the first bio-chip 100 including the first culture medium 310 and the bio-material 330 and a second bio-chip 200 accommodating a second culture medium 320 therein may be prepared. In detail, in the operation, the first bio-chip 100 requiring the replacement of the culture medium and the second bio chip 200 in which a new culture medium is accommodated may be prepared.

The first bio-chip 100 may include the bio-material 330 and require the replacement of the first culture medium 310.

The first bio-chip 100 may include a plurality of recesses each accommodating the first culture medium 310 and the bio-material 330 therein. The first bio-chip 100 may have a shape identical or similar to that of the bio-chips illustrated in FIGS. 1 and 2.

The second bio-chip 200 may be a chip which does not include the bio-material 330 and includes the second culture medium 320, a new culture medium. The second bio-chip 200 may have the same shape as that of the first bio-chip 100.

(Inverting the First Bio Chip)

In this operation, the first bio-chip 100 in which the bio-material 330 is accommodated may be inverted vertically. In detail, in the operation, the first bio-chip 100 may be vertically inverted to allow the bio-material 330 included in the first culture medium 310 to be leaked from the first culture medium 310.

In general, the bio-material 330 included in the first culture medium 310 has a large mass in comparison to that of the first culture medium 310, so the bio-material 330 may be sunk to the bottom of each recess 112.

However, when the first bio-chip 100 is vertically inverted, the bio-material 330 moves in a gravitation direction to be transferred to a surface layer of the first culture medium 310. Thereafter, although not shown, the bio-material 330 may be leaked out of the first culture medium 310 by lapse of a predetermined time.

In view of the fact, the bio-material 330 of the first culture medium 310 may be transferred to the second culture medium 320 without a separate instrument.

(Bonding and Separating the First and Second Bio-Chips)

In this operation, the vertically inverted first bio-chip 100 and the second bio-chip 200 may be bonded.

When the bio-material 330 of the first culture medium 310 moves in the gravitation direction to a certain degree through the vertical inversion of the first bio chip 100, the first bio chip 100 and the second bio-chip 200 may be bonded.

Here, the bonding of the first bio-chip 100 and the second bio-chip 200 may refer to allowing the two bio-chips to approach nearer to each other such that the first culture medium 310 and the second culture medium 320 are brought into contact with each other, rather than physically joining the two bio-chips.

Meanwhile, when the first culture medium 310 and the second culture medium 320 are brought into contact with each other, contact between the culture mediums may be undertaken within a short time (within one second) and then the culture mediums may be separated such that mixture of the culture mediums is minimized. Here, the bio-material 330 may move to the second culture medium 320 of the bio-chip 200 positioned below in the process of separating the bio-chips 100 and 200, by gravitation.

The method of replacing a culture medium including the foregoing operations does not require a separate instrument, so culture medium replacement may be easily undertaken.

Also, in the method of replacing a culture medium, according to the embodiment, since the bio-material 330 provided in plural and accommodated in the first bio-chip 100 may be simultaneously moved to the second bio-chip 200, the operation of replacing the culture medium can be quickly performed.

Meanwhile, in order to promote or accelerate the movement of the bio-material of the first culture medium 310 to the second culture medium 320, the first bio-chip 100 may be vibrated.

A method of replacing a culture medium of the bio-chip according to the second embodiment of the present invention will be described with reference to FIGS. 8 through 10.

A method of replacing a culture medium of the bio-chip according to the second embodiment of the present invention is different from that of the first embodiment, in that the auxiliary member 130 is used.

In order to allow the bio-material to be smoothly cultured, the bio-chip may store a sufficient amount of a culture medium. In this case, in a similar manner to the first embodiment as mentioned above, the culture medium protrudes upwardly from the surface of the bio-chip so as to come into contact with the other culture medium.

However, in some cases, the culture medium may not be stored to a degree sufficient to completely fill the recesses of the bio-chip or the recesses may have a significant depth.

In this case, although the bio-chip is inverted vertically, the culture medium may not be protruded to the outside of the recesses, so the culture medium may not be replaced through the case of the first embodiment.

The method of replacing a culture medium according to the embodiment is to resolve the defect.

The method of replacing a culture medium, according to the present invention may include preparing the first and second bio-chips; inserting the auxiliary member; inverting the first bio-chip; and bonding and separating the first and second bio-chips. For reference, the operations other than the inserting of the auxiliary member, are the same as those of the first embodiment, so detailed descriptions thereof will be omitted.

(Inserting the Auxiliary Member)

In this operation, the first bio-chip 100 in which the first culture medium 310 and the bio-material 330 are accommodated and the auxiliary member 130 may be coupled.

The auxiliary member 130 may have a shape as shown in FIG. 3 or 5 and may reduce an effective volume of the recesses 112. Namely, the auxiliary member 130 may include the insertion pillars 132 inserted in the recesses 112.

Accordingly, when the first bio-chip 100 and the auxiliary member 130 are coupled, the first culture medium 310 stored in the recesses 112 may protrude convexedly and upwardly from the surface of the auxiliary member 130.

When the first culture medium 310 protrudes convexedly, the operation of vertically inverting the first bio chip 100 and the operation of bonding and separating the first bio-chip 100 and the second bio-chip 200 are performed to transfer the bio-material from the first bio-chip 100 to the second bio-chip 200.

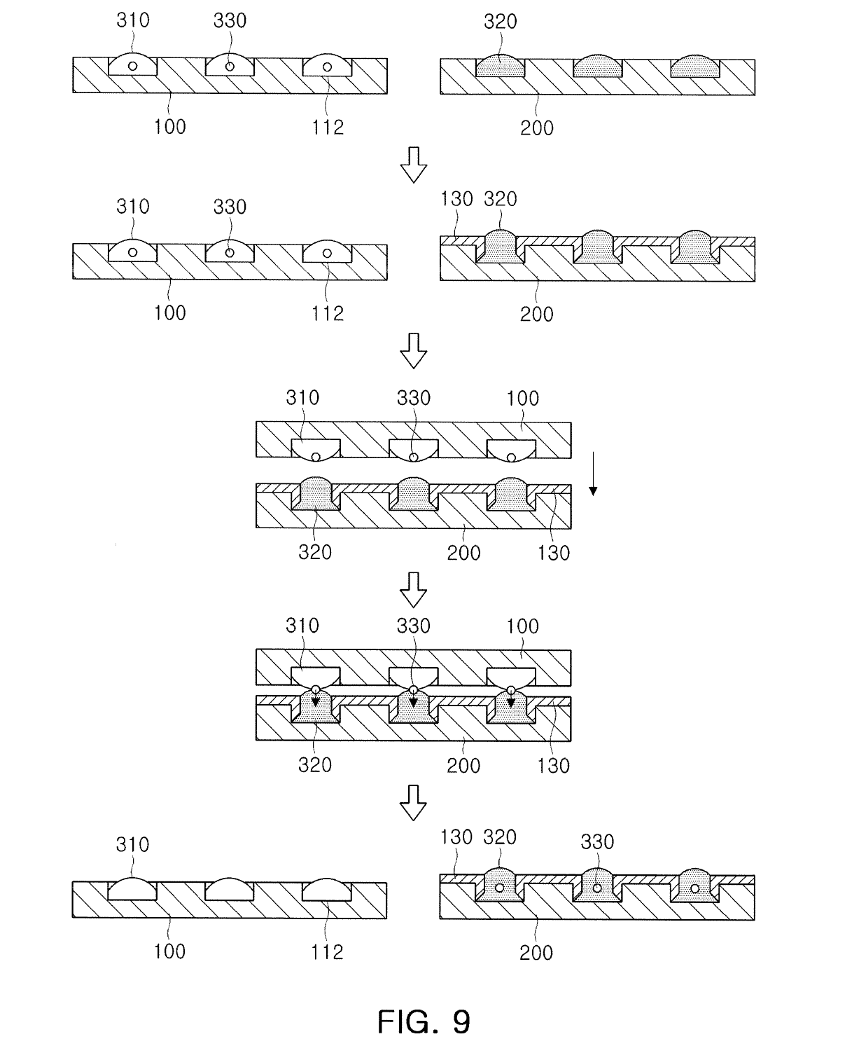

Meanwhile, as shown in FIG. 9, the auxiliary member 130 may be mounted on the second bio-chip 200 storing the second culture medium 320 therein or may be mounted in both of the first bio-chip 100 and the second bio-chip 200.

As set forth above, according to the embodiments of the invention, the bio-material included in an old culture medium can be easily moved to a new culture medium without being damaged.

While the present invention has been shown and described in connection with the embodiments, it will be apparent to those skilled in the art that modifications and variations can be made without departing from the spirit and scope of the invention as defined by the appended claims.

What is claimed is:

1. A bio-chip comprising:
a substrate member including a plurality of recesses formed therein to accommodate a culture medium; and
space maintaining members formed on the substrate member and maintaining a space between the substrate member and another substrate member to allow a bio-material in the culture medium to be transferred to a culture medium of another substrate member.

2. The bio-chip of claim 1, wherein the space maintaining members are asymmetrical with respect to a bisecting line of the substrate member in a first length direction or a second length direction thereof.

3. The bio-chip of claim 2, wherein each space maintaining member includes:
a first portion having a convex shape; and
a second portion having a concave shape to be engaged with the first portion having the convex shape.

4. The bio-chip of claim 1, wherein each recess includes:
a bottom face coated with a hydrophilic material; and
a side face coated with a hydrophobic material.

5. The bio-chip of claim 1, wherein the space maintaining members are disposed in a circular manner, based on the recesses.

6. A bio-chip comprising:
a substrate member including a plurality of recesses formed therein to accommodate a culture medium; and
an auxiliary member including a plurality of insertion pillars inserted into the recesses to allow the culture medium to be raised upwardly from the recesses, and through holes connected to the recesses.

7. The bio-chip of claim 6, wherein each recess has a bottom face coated with a hydrophilic material.

8. The bio-chip of claim 6, wherein the auxiliary member is coated with a hydrophobic material.

9. The bio-chip of claim 6, wherein each insertion pillar has a cylindrical shape having a hole connected to each recess.

10. The bio-chip of claim 9, wherein each insertion pillar has a cross-sectional shape the same as that of each recess.

11. The bio-chip of claim 9, wherein each insertion pillar is coated with a hydrophobic material.

12. The bio-chip of claim 9, wherein each insertion pillar has a sloped face formed at an end thereof.

13. A method of replacing a culture medium of a bio-chip, the method comprising:
preparing a first bio-chip accommodating a first culture medium, an old culture medium, therein and a second bio-chip accommodating a second culture medium, a new culture medium, therein;
vertically inverting the first bio-chip to allow a bio-material included in the first culture medium to move out of a surface of the first culture medium;
allowing the first and second bio-chips to approach nearer to each other such that the first and second culture mediums are brought into contact with each other; and
separating the first and second bio-chips when the bio-material moves to the second culture medium through contact between the culture mediums.

14. The method of claim 13, further comprising vibrating the first bio-chip such that the bio-material in the first culture medium easily moves into the second culture medium.

15. The method of claim 13, further comprising disposing a space maintaining unit between the first and second bio-chips such that a predetermined distance is maintained between the first and second bio-chips.

16. A method of replacing a culture medium of a bio-chip, the method comprising:
preparing a first bio-chip including a first recess formed therein and accommodating a first culture medium, an old culture medium, and a second bio-chip including a second recess formed therein and accommodating a second culture medium, a new culture medium;
reducing an effective volume of the first recess such that the first culture medium is raised upwardly from a surface of the first bio-chip;
vertically inverting the first bio-chip such that the bio-material included in the first culture medium moves out of a surface of the first culture medium;
allowing the first and second bio-chips to approach nearer to each other such that the first and second culture mediums are brought into contact with each other; and
separating the first and second bio-chips when the bio-material moves to the second culture medium through contact between the culture mediums.

17. The method of claim 16, wherein in the reducing of the effective volume of the first recess, an auxiliary member having an insertion member inserted into the first recess and a through hole connected to the first recess is stacked on the first bio-chip.

18. The method of claim 16, further comprising:
reducing an effective volume of the second recess such that the second culture medium of the second bio- chip is raised upwardly from a surface of the second bio-chip.

19. The method of claim 18, wherein in the reducing of the effective volume of the second recess, an auxiliary member having an insertion member inserted into the second recess and a through hole connected to the second recess is stacked on the second bio-chip.

20. A method of replacing a culture medium of a bio-chip, the method comprising:
preparing a first bio-chip including a first recess formed therein and accommodating a first culture medium, an old culture medium, and a second bio-chip including a second recess formed therein and accommodating a second culture medium, a new culture medium;
coupling an auxiliary member having an insertion pillar to be inserted into the second recess to the second bio-chip such that the second culture medium is raised upwardly from a surface of the second bio-chip;
vertically inverting the first bio-chip such that the bio-material included in the first culture medium moves out of a surface of the first culture medium;
allowing the first and second bio-chips to approach nearer to each other such that the first and second culture mediums are brought into contact with each other; and
separating the first and second bio-chips when the bio-material moves to the second culture medium through contact between the culture mediums.
